SEQUENCE LISTING (3 sequences)

SEQ ID NO: 1 - protein, 555 aa, Homo sapiens [sequence omitted]

SEQ ID NO: 2 - protein, 576 aa, Mus musculus
MAAAAAAAAIGYRGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRILQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRPQKELVHLSELPPTPLHISEDSSSPERTVPQDSSVIATLVDQATNNNVNDLEVSESSCVEEKPQEVQTSRNVENINVENPDFKAVGSSTADKNERTSVAETVRKCWPNRLAKEQISKRLEGNQYLFVPPNRYIFHGAEVYSDSEDDVLSSSSCGSNSDSGTCQSPSLEEPLEDESEIEEFYNGLEDDTERPECAGGSGFGADGGDQEVVNEAIATRQELTDVNYPSDKS

SEQ ID NO: 3 - DNA, 5420 bp, Artificial sequence
  plasmid DNA
agcgcctgatgcggtattttctccttacgcatctgtgcggtatttcacaccgcatatcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacagatgtgatatggctgattatgatcattacttatctaggtataggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgaatttagcggccgcgaattcgcccttgtctagagtggaacaattcctgtacctgcacaattattacactatgatttgtttgatggatagttcatgtctgttacttcctgtttcacagatatagcttcattaattgcctcttgatcatctccatcagtcccaaatccagctcctccagctctctctggaacatcaggctcatcttctaagccattgtagaattcttcaatttcactttcatcctccatgggttcttctaaacttggactctggcatgtcccactatcactgttactgccacaagaactagaggataagacgtcatcttcagagtctgaatatacctcagcgccatggaaaatgtaacgatttggtggcaaaaacagatactgattaccatcaagccgcctactaatctgctcctttgccactctattaggccagcattttctcactgttccagccactgaagttctttcatttttctccccagtactagaaccaacattcttcaaatccggattttccatctgttcagcaatactttcaacattcctagaagtttgtacttcctgtggtttttcttccatacaaccttttgattcagacacatctaaatcatcattactcttagctgcttggtctaaaagtgtgacaatcactgaagaatctggtggtgaagttctttctggtgaacttgagtcttctgaaacatgaagaggtgtgggtggcaactctgacaaataagccaattctttttgtgttcgtggaggtttttcagtaatttctgaaagctttacagggttacagcaaagtttggcatattcaccacctaacctatgacacaattcattaattatgacatcacagtctccaagaagctctacatcaaaatgcagatgaggcaaaggttctctattaattaatatctgaggcacttcatggggtatggaacttggaattagtgctactggtcttactttgagggaagacccaataacaatgaggaggtcaacttcatctttgtcatacttcatggctctatgaaactgttctggtaaattttcaccaaaaaacacaatctctggtttcatgatagcaagcggttcatcagctgggcacctaggacatcgaggaactacctgattaaaaatatctcctcgtacagcttcacagtcaactttgtatttacaaatcaggcaagatgctgttgcaaaggaaccatgacactgaattatcctttggattcccgcaacctgttccagcgtgtctatgttctgggtatagttgcgaagtagttttccttccttatctgacaaggctatgaatttgtgacagagagatggctggaattgtccaggatatatttcctttgcaaacttgaagaatggtcttggatcttttctgaaatattcaatatcaaacatcgcttgaggatctggaagatctgggaagtctacagcaaggcgagcataaataccatcccttgacctgaagtcaggtattccacatgaaacagacaccccagctccagttagaactataatttttttgcactcttgcagtaatttcacagcatcttcaattgtattaatatcttttctttttttcctttttggtggttctgaaaggatattaataacaatctgccacagtgtcatatcatccaactcaggtggaggtattgtttccggcaataaatctttaagaattgttcgaggatctgtgccaatcataagatgttgctgaacaaaagtatatggacctatccgtggccttggagtccagtcactagagcttgcatgtgaggctctatcctcctcatcactttcacaggaatgaaaaccattagtgataatttcatcaccgaacagaaggttatctcggtacccaatcgccgccgccgccgcctcttcctcctcctcgccctcgtcgtcgtcgtcttcgtcgtacaagttgtcggccagcggtggctcccgagatgggccctgcaggcccggcccattgtctccttccccagccgccgcagtcgcctgggcctcttgctccccgcctgccgccgccgcctctgcctccgcctcccgccacagcgccgccgccgccgcacccgggcagcccctggccgccgccggcacctcacgctctggggccgccccaccgggctcgcccgggctccgctcgaggccgggaccatctctccgcggcctcttgcggagcggctccccggcgggggacgacgcggcctccctgtcggcccccgccgccgagggggagccgccgggctgaagggcgagggccgcctcgtccgccgaattcccgggtttatcgtcatcgtccttgtagtccatcctgaggatccgagctcggtaccaagcttagatctcctccaaaaaagcctcttcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccttgctgactaattgagatgcatgctttgcatacttctgcctgctggggagcctggggactttccacaccctaactgacacacattccacagctggttctttccgcctcagaaggtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

(19) United States
(12) Patent Application Publication
(10) Pub. No.: US 2018/0201899 A1
(43) Pub. Date: Jul. 19, 2018

(54) IPSC-EC PERFORMANCE ENHANCEMENT VIA SIRT1 OVEREXPRESSION

(71) Applicant: Northwestern University, Evanston, IL (US)

(72) Inventors: Guillermo A. Ameer, Chicago, IL (US); Bin Jiang, Evanston, IL (US); Michele Jen, Dallas, TX (US); Jason A. Wertheim, Evanson, IL (US)

(21) Appl. No.: 15/744,216

(22) PCT Filed: Jul. 15, 2016
(86) PCT No.: PCT/US2016/042496

§ 371 Date: Jan. 12, 2018

Related U.S. Application Data
(60) Provisional application No. 62/192,903, filed on Jul. 15, 2015.

(51) Int. Cl. C12N 5/071; A61K 35/44; A61K 38/50; C12N 9/80; G01N 33/50

(52) U.S. Cl. CPC C12N 5/069; A61K 35/44; A61K 38/50; C12N 2506/45; G01N 33/502; G01N 33/5014; C12N 9/80

(57) ABSTRACT

Compositions comprising endothelial cells (ECs) differentiated from induced pluripotent stem cells (iPSC)that over-express Sirtuin 1 (SIRT1) are disclosed. Further disclosed are methods of preparation of the compositions, and methods for treating a subject comprising administering transplatanbe cells, tissue, or organ comprising the iPSC-derived ECs overexpressing SIRT1, as well as methods of testing an agent for therapeutic efficacy and toxicity using the compositions.

### CROSS-REFERENCE TO RELATED APPLICATIONS

The present invention claims the priority benefit of U.S. Provisional Patent Application 62/192,903, filed Jul. 15, 2015, which is incorporated by reference in its entirety.

### STATEMENT REGARDING FEDERAL FUNDING

This invention was made with government support under K08 DK101757 and R01 EB017129 awarded by the National Institutes of Health. The government has certain rights in the invention.

### FIELD

Compositions comprising endothelial cells (ECs) differentiated from induced pluripotent stem cells that overexpress Sirtuin1 (SIRT1), and methods of use and preparation thereof.

### BACKGROUND

Endothelial cells (ECs) that are differentiated from induced pluripotent stem cells (iPSCs) can be used in establishing disease models for personalized drug discovery or developing patient-specific, vascularized tissues or organoids. However, a number of technical challenges are often associated with iPSC-ECs in culture, including instability of the endothelial phenotype and limited cell proliferative capacity over time. Early senescence is believed to be the primary mechanism underlying these limitations.

### SUMMARY

In some embodiments, provided herein are compositions comprising endothelial cells (ECs) differentiated from induced pluripotent stem cells that overexpress Sirtuin1 (SIRT1). In some embodiments, provided herein are compositions comprising induced pluripotent stem cell (iPSC)-derived endothelial cells (ECs) that overexpress Sirtuin1 (SIRT1). In some embodiments, the iPSC-derived ECs comprise exogenous nucleic acid encoding SIRT1. In some embodiments, the exogenous nucleic acid encodes a polypeptide having at least 70% (e.g., 70%, 75%, 80%, 85%, 90%, 95%, 96%, 97%, 98%, 99%, 100%, or ranges therebetween) sequence identity with all or a portion of wild-type human SIRT1 (SEQ ID NO: 1). In some embodiments, the exogenous nucleic acid encodes a polypeptide having at least 70% (e.g., 70%, 75%, 80%, 85%, 90%, 95%, 96%, 97%, 98%, 99%, 100%, or ranges therebetween) sequence identity with all or a portion of SEQ ID NO: 2. In some embodiments, the exogenous nucleic acid encoding SIRT1 is within an expression vector (e.g., SEQ ID NO: 3). In some embodiments, the expression vector is a viral vector. In some embodiments, the expression vector is a lentiviral vector.

In some embodiments, provided herein are methods of maintaining endothelial cell (EC) phenotype, improving EC function, enhancing proliferative capacity, and/or overcoming early senescence in induced pluripotent stem cell (iPSC)-derived ECs, comprising overexpressing Sirtuin1 (SIRT1) in said iPSC-derived ECs. In some embodiments, overexpressing SIRT1 in said iPSC-derived ECs comprises tranducing, transfecting, or transforming a SIRT1-encoding vector into the iPSC-derived ECs. In some embodiments, the SIRT1-encoding vector is tranduced, transfected, or transformed into the iPSC-derived ECs after passage 1, after passage 2, after passage 3, after passage 4, after passage 5, after passage 6, after passage 7, after passage 8, after passage 9, after passage 10, etc.

In some embodiments, provided herein are methods comprising: (a) inducing theformation of pluripotent stem cells (iPSCs) from non-pluripotent somatic cells; (b) differentiating the iPSCs into iPSC-derived endothelial cells (ECs); and (c) overexpressing Sirtuin1 (SIRT1) in said iPSC-derived ECs.

In some embodiments, provided herein are disease models comprising endothelial cells (ECs) differentiated from induced pluripotent stem cells that overexpress Sirtuin1 (SIRT1).

In some embodiments, provided herein are transplantable cells, tissue, or organ comprising the iPSC-derived ECs overexpressing SIRT1 described herein.

In some embodiments, provided herein are methods of treating a subject comprising administering the the transplatanbe cells, tissue, or organ comprising the iPSC-derived ECs overexpressing SIRT1 described herein to a subject.

In some embodiments, provided herein are methods of testing an agent comprising administering the agent to a composition comprising the iPSC-derived ECs overexpressing SIRT1 described herein. In some embodiments, the agent is testing for therapeutic efficacy. In some embodiments, the agent is testing for toxicity.

### BRIEF DESCRIPTION OF THE DRAWINGS

FIG. 1. Change in morphology and phenotype of iPSC-ECs from passage 1 (A) to passage 6 (F), with representative flow cytometry data from passage 1 (G) and passage 5 (H). Phase contrast images (A-F) show that cells gradually lose the cobble stone-like morphology over time and take on a fibroblast-like appearance. Expression of EC markers (CD31 and CD144) gradually decreased over time. Scale bar=100 μm. 138×167 mm.

FIG. 2. Effect of empty (A, D, G), SIRT1H363Y (B, E, H) and SIRT1 (C, F, I) lentiviral transduction on iPSC-ECs at passage 5, including phase contrast images of iPSC-ECs in culture for morphological assessment (A-C); immunofluorescent staining for SIRT1 (green) as an indication of transduction efficiency (D-F); and flow cytometry analysis for putative markers (x axis: CD31; y axis: CD144) of EC phenotype (G-I). 97×83 mm).

FIG. 3. Cellular senescence associated β-galactosidase (β-gal) staining (blue) of iPSC-EC at passage 6 for empty (A, D), SIRT1H363Y (B, E) and SIRT1 (C, F) lentiviral transduction in the absence (A-C) and presence (D-F) of Ex-527, a SIRT1 inhibitor. 113×58 mm.

FIG. 4. Functional assessment of iPSC-EC with or without viral transduction for (A) HDAC activity, (B) proliferation, (C) response to VEGF and (D) nitric oxide production. * indicates p<0.05 (n=4). 89×70 mm.

FIG. 5. Flow cytometry analysis of CD 31 expression (FITC-A) for iPSC-ECs with LV-SIRT1 at passages 5 (A), 7 (B) and 9 (C). The percentages of CD 31 positive population (grey histogram) are shown compared to isotype control (white histogram).

FIG. 6. Tube formation assay for iPSC-EC at passage 6 with empty (A), SIRT1^{H363Y} (B) and SIRT1 (C) lentiviral transduction. Fluorescent microscopic imaging was taken 4 hours after cell seeding with Calcein AM staining. Scale bar=100 μm.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “SIRT1-expressing endothelial cell” is a reference to one or more SIRT1-expressing endothelial cells, unless the context clearly dictates otherwise.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein the term “stem cell” (“SC”) refers to cells that can self-renew and differentiate into multiple lineages. A stem cell is a developmentally pluripotent or multipotent cell. A stem cell can divide to produce two daughter stem cells, or one daughter stem cell and one progenitor (“transit”) cell, which then proliferates into the tissue's mature, fully formed cells. Stem cells may be derived, for example, from embryonic sources (“embryonic stem cells”) or derived from adult sources. For example, U.S. Pat. No. 5,843,780 to Thompson describes the production of stem cell lines from human embryos. PCT publications WO 00/52145 and WO 01/00650 describe the use of cells from adult humans in a nuclear transfer procedure to produce stem cell lines.

As used herein, the term “pluripotent cell” or “pluripotent stem cell” refers to a cell that has complete differentiation versatility, e.g., the capacity to grow into any of the mammalian body's approximately 260 cell types. A pluripotent cell can be self-renewing, and can remain dormant or quiescent within a tissue. Unlike a totipotent cell (e.g., a fertilized, diploid egg cell), a pluripotent cell, even an pluripotent embryonic stem cell, cannot usually form a new blastocyst.

As used herein, the term “induced pluripotent stem cells” (“iPSCs”) refers to a stem cell induced from a somatic cell, e.g., a differentiated somatic cell, and that has a higher potency than said somatic cell. iPS cells are capable of self-renewal and differentiation into mature cells (e.g., endothelial cells).

As used herein the term “feeder cells” refers to cells used as a growth support in some tissue culture systems. Feeder cells may be embryonic striatum cells or stromal cells. As used herein, the term “endothelium” refers to a layer of cells that line the inside surfaces of blood vessels and form capillaries. The term “endothelial cell” refers to the specialized cells that form the epithelium and line the inner walls of blood vessels.

As used herein, the term “overexpress” refers to increasing the expression of a protein to a level greater than the cell normally produces. It is intended that the term encompass overexpression of endogenous, as well as exogenous proteins.

As used herein, the term “transduction” refers to the introduction of nucleic acid sequences into a eukaryotic cell by a replication-defective retrovirus.

As used herein, the term “transfection” refers to the introduction of foreign or exogenous DNA by a cell. A number of transfection techniques are well known in the art, see, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al., 1981, Gene 13:197. Such techniques can be used to introduce one or more exogenous DNA moieties into suitable host cells. Depending on the technique used to make the transfected cell and the desired use of the transfected cell, a cell can be transfected either stably or transiently.

As used herein, the term “endogenous” refers to material (e.g., nucleic acids, polypeptides, etc.) that is native to (e.g., within the natural genome of, encoded by the natural genome of) a cell, or cell type, and does not originate from outside of the cell or the the cell's lineage.

As used herein, the term “exogenous” refers to to material (e.g., nucleic acids, polypeptides, etc.) that is not native to a cell or cell type and is instead introduced to the cell or the cell's lineage using synthetic, recombinant, and/or genetic engineering methods.

### DETAILED DESCRIPTION

Compositions comprising endothelial cells (ECs) differentiated from induced pluripotent stem cells that overexpress Sirtuin1 (SIRT1), and methods of use and preparation thereof.

Induced pluripotent stem cells (iPSCs) are a non-natural cell source for disease modeling (refs.1-2; herein incorporated by reference in their entireties), drug discovery (refs.3,4; herein incorporated by reference in their entireties), and potentially patient-specific tissue regeneration (refs.5,6; herein incorporated by reference in their entireties). Specifically, endothelial cells (ECs) that are derived from iPSCs could be used in vascular repair and regeneration (ref 7; herein incorporated by reference in its entirety). However, early senescence, limited cell proliferation and instability of the endothelial phenotype remain significant challenges to the large scale production and wide-scale use of these cells (refs.8,9; herein incorporated by reference in their entireties). Therefore, strategies need to be developed to improve the durability and performance of iPSC-EC in culture.

Sirtuin 1 (SIRT1) is a nicotinamide adenine dinucleotide (NAD⁺)-dependent histone deacetylase (HDAC) that functions in mammalian cells to promote cell survival (ref.10; herein incorporated by reference in its entirety) and prevent stress induced senescence (ref.11; herein incorporated by reference in its entirety). Moreover, SIRT1 plays various roles in maintaining endothelial function, including angiogenesis via deacetylation of the forkhead transcription factor (FOXO1) (ref.12; herein incorporated by reference in its entirety); nitric oxide (NO) production via deacetylating and activating endothelial nitric oxide synthase (eNOS) (ref.13; herein incorporated by reference in its entirety); cell proliferation by targeting the LKB1-AMPK pathway (ref.14; herein incorporated by reference in its entirety); and inhibiting oxidative stress via p53 deacetylation (refs.15,16; herein incorporated by reference in their entireties).

Sirtuin1 (SIRT1) is an NAD⁺ dependent deacetylase involved in the regulation of cell senescence, redox state, and inflammatory status. Experiments conducted during development of embodiments herein demonstrate that overexpression of the SIRT1 gene in iPSC-ECs maintains EC phenotype, function, and proliferative capacity by overcoming early cell senescence. The SIRT1 gene was packaged into a lentiviral vector (LV-SIRT1) and transduced into iPSC-ECs at passage 4. Beginning with passage 5, iPSC-ECs exhibited a fibroblast-like morphology whereas iPSC-ECs overexpressing SIRT1 maintained EC cobblestone morphology. SIRT1 overexpressing iPSC-ECs also exhibited a higher percentage of canonical markers of endothelia (LV-SIRT1 61.8% CD31+ vs. LV-empty 31.7% CD31+, p<0.001; LV-SIRT1 46.3% CD144+ vs. LV-empty 20.5% CD144+, p=0.011), with higher nitric oxide synthesis, lower β-galactosidase production indicating decreased senescence (3.4% for LV-SIRT1 vs. 38.6% for LV-empty, p<0.001), increased deacetylation activity, and higher proliferation rate. SIRT1 overexpressing iPSC-ECs continued to proliferate through passage 9 with high purity of ECs while iPSC-ECs without SIRT1 overexpression became senescent after passage 5. SIRT1 overexpression in iPSC-ECs maintains EC phenotype, improves EC function and extends cell lifespan, overcoming critical hurdles associated with the use of iPSC-ECs in translational research.

Embodiments herein relate to induced pluripotent stem cells (iPSCs). In particular, embodiments herein related to endothelial cells (ECs) derived from (differentiated from) iPSCs.

In some embodiments, iPSCs are induced using polypeptides exogenous polypepides and/or nucleic acids. The polypeptides (or nucleic acids encoding such polypeptides) used to induce the formation of iPSCs may include any combination of Oct3/4 polypeptides, Sox family polypeptides (e.g., Sox2 polypeptides), KIf family of polypeptides (e.g., Klf4 polypeptides), Myc family polypeptides (e.g., c-Myc), Nanog polypeptides, Lin28 polypeptides, and others understood in the field to be useful for generating iPSCs. For example, in some embodiments, nucleic acid vectors designed to express Oct3/4, Sox2, Klf4, Lin28, and/or c-Myc polypeptides are used to obtain induced pluripotent stem cells. In some cases, polypeptides are directly delivered into target cells to obtain induced pluripotent stem cells using a polypeptide transfection method (e.g., liposome or electroporation). In other embodiments, nucleic acid vectors designed to express iPSC-inuding polypeptides (e.g., Oct3/4, Sox2, Klf4, Lin28, and/or c-Myc polypeptides), are used to obtain induced pluripotent stem cells. Methods and reagents (e.g., polypeptides) for inducing the formation of pluripotent stem cells are not limited to the above, and additional methods and reagents understood in the field are within the scope herein.

In some embodiments, any appropriate cell type is used to obtain induced pluripotent stem cells. In some embodiments, skin, lung, heart, liver, blood, kidney, muscle cells, etc. are used to obtain iPSCs. Such cells can be obtained from any type of mammal including, without limitation, humans, mice, rats, dogs, cats, cows, pigs, or monkeys. In addition, any stage of the mammal can be used, including mammals at the embryo, neonate, newborn, or adult stage.

In some embodiments, methods and reagents herein are used to differentiate iPSCs into endothelial cells (ECs). For example, in some embodiments, to initiate differentiation, iPSCs are incubated type IV collagenase and cultured (e.g., in ultra low attachment dishes) in differentiation media to form embryoid bodies. Exemplary differentiation media may comprise α-Minimum Eagle's Medium, fetal bovine serum, L-glutamine, β-mercaptoethanol, non-essential amino acids, bone morphogenetic protein-4 (BMP-4), vascular endothelial growth factor (VEGF-A), etc. In some embodiments, for arterial EC differentiation, EBs (e.g., 4-day EBs) are then seeded (e.g., on gelatin-coated dishes) and cultured (e.g., in the presence of VEGF-A and 8-bromoadenosine-3′:5′-cyclic monophosphate sodium salt). In some embodiments, for venous EC differentiation, the EBs (e.g., 4-day EBs) are differentiated in VEGF-A. In some embodiments, for lymphatic EC differentiation, EBs (e.g., 4-day EBs) are differentiated in BMP-4, VEGF-A, VEGF-C, and angiopoietin-1. Other methods and reagents for the differentiation of pluripotent stems cells, or induced pluripotent stem cells, into endothelial cells are understood in the field, within the scope herein, and described, for example, in Lian et al. Stem Cell Reports. 2014 Nov. 11; 3(5): 804-816; Yoder. Curr Opin Hematol. 2015 May; 22(3): 252-257; WO 2013/166165; WO 2014/200340; Bernardini et al. J Biol Chem. 2014 Feb. 7; 289(6):3383-93; each of which are herein incorporated by reference in their entireties. Such methods and reagents find use in embodiments herein.

In some embodiments, any suitable methods and vectors are used to introduce nucleic acid (e.g., to induce formation if IPSCs, to differentiate iPSCs into ECs, to introduce SIRT1, etc.) into a cell. In some embodiments, nucleic acid encoding polypeptides are transferred to the cells using: recombinant viruses that infect cells, liposomes, other non-viral methods such as electroporation, microinjection, transposons, phage integrases, or calcium phosphate precipitation, that are capable of delivering nucleic acids to cells. In some embodiments, an exogenous nucleic acid is delivered as part of a vector in which a regulatory element such as a promoter is operably linked to the nucleic acid of interest (e.g., to induce formation if IPSCs, to differentiate iPSCs into ECs, to introduce SIRT1, etc.). In some embodiments, the promoter is constitutive or inducible. Non-limiting examples of constitutive promoters include cytomegalovirus (CMV) promoter and the Rous sarcoma virus promoter. An inducible promoter is a promoter that is capable of directly or indirectly activating transcription of one or more DNA sequences or genes in response to an inducer. In the absence of an inducer, the DNA sequences or genes will not be transcribed. In some embodiments, the inducer is a chemical agent such as a protein, metabolite, growth regulator, phenolic compound, or a physiological stress imposed directly by, for example heat, or indirectly through the action of a pathogen or disease agent such as a virus. In some embodiments, additional regulatory elements are present in a vector, such as polyadenylation sequences, translation control sequences (e.g., an internal ribosome entry segment, IRES), enhancers, or introns. In some embodiments, vectors also include other elements. For example, in some embodiments, a vector includes a nucleic acid that encodes a signal peptide such that the encoded polypeptide is directed to a particular cellular location (e.g., the cell surface) or a nucleic acid that encodes a selectable marker. Non-limiting examples of selectable markers include puromycin, adenosine deaminase (ADA), aminoglycoside phosphotransferase, dihydrofolate reductase (DHFR), hygromycin-B-phosphtransferase, thymidine kinase (TK), and xanthin-guanine phosphoribosyltransferase (XGPRT). Such markers are useful for selecting stable transformants in culture.

In some embodiments, any appropriate viral vector is used to introduce nucleic acids (e.g., to induce formation if IPSCs, to differentiate iPSCs into ECs, to introduce SIRT1, etc.). Examples of viral vectors include, without limitation, vectors based on DNA or RNA viruses, such as adenovirus, adeno-associated virus (AAV), retroviruses, lentiviruses, vaccinia virus, measles viruses, herpes viruses, baculoviruses, and papilloma virus vectors. See, Kay et al., Proc. Natl. Acad. Sci. USA, 94:12744-12746 (1997) for a review of viral and non-viral vectors; incorporated by reference in its entirety. In some embodiments, viral vectors are modified so the native tropism and pathogenicity of the virus has been altered or removed. The genome of a virus also can be modified to increase its infectivity and to accommodate packaging of the nucleic acid encoding the polypeptide(s) of interest (e.g., to induce formation if IPSCs, to differentiate iPSCs into ECs, to introduce SIRT1, etc.).

In some embodiments, appropriate non-viral vectors are used to introduce nucleic acids (e.g., to induce formation if IPSCs, to differentiate iPSCs into ECs, to introduce SIRT1, etc.). Examples of non-viral vectors include, without limitation, vectors based on plasmid DNA or RNA, retroelement, transposon, and episomal vectors. In some embodiments, non-viral vectors are delivered to cells via liposomes, which are artificial membrane vesicles. The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Transduction efficiency of liposomes can be increased by using dioleoylphosphatidylethanolamine during transduction. See, Feigner et al., J. Biol. Chem., 269:2550-2561 (1994); incorporated by reference in its entirety. High efficiency liposomes are commercially available.

In some embodiments, iPSCs and iPSC-derived ECs are obtained using culture conditions that do not involve the use of serum or feeder cells. In some embodiments, iPSCs and iPSC-derived ECs are obtained using culture conditions that involve the use of serum or feeder cells.

Embodiments herein find use in, for example, in vitro disease modeling with iPSC-ECs from cardiovascular diseases, vascular tissue engineering using iPSC-EC, neovascularization for tissue ischemia using iPSC-EC, etc.

### Experimental

### EXAMPLE 1

### Materials and Methods

### Lentivirus (LV) Construction

SIRT1 (Addgene plasmid 1791) plasmid DNA were cloned into a lentiviral transfer vector, pWPI. Lentiviral packaging vectors, pMD2.g and psPAX2, were co-transfected with pWPI, pWPI-SIRT1 (mass ratio 1:3:4, respectively) into HEK-293FT cells using Fugene HD at 3:1 total DNA mass to Fugene HD (Promega, Madison, Wis.) volume ratio complexed in Opti-MEM (Life Technologies, Carlsbad, Calif.). After 48 h of transfection, the supernatant was collected and purified using Lenti-X Maxi Purification Kit (ClonTech, Mountain View, Calif.) and subsequently concentrated using Lenti-X concentrator (ClonTech, Mountain View, Calif.). The lentivirus titer was determined using a qPCR lentivirus titration kit (Applied Biological Materials, Richmond, BC, Canada).

Transduction to iPSC-ECs

Lentivirus with SIRT1 at MOI 5 were added to iPSC-EC at the end of passage 4 for 48 hours. Transduction efficiency was evaluated by quantifying the percentage of cells stained positive for human SIRT1 (Santa Cruz, Dallas, Tex.). At the end of each passage, iPSC-ECs were trypsinized and stained for EC markers CD31 (PECAM) and CD144 (VE-Cadherin) using FITC-conjugated CD31 antibody (Sigma-Aldrich, St Lois, Mo.) and PE-conjugated CD144 antibody (Life Technologies, Carlsbad, Calif.). Flow cytometry was performed using BD LSR II flow cytometer (San Jose, Calif.) and the data analyzed with FlowJo (Ashland, Oreg.).

A 60.3±7.3% transduction efficiency was measured via immunohistomorphometry of SIRT1 positive cells. Cells overexpressing SIRT1 exhibit a higher degree of EC-like cobblestone morphology compared to the control groups. Expression of EC markers such as CD31 and CD144 was also significantly higher in the SIRT1 overexpressed group (61.8±3.6% CD31+, 46.3±9.7% CD144+) relative to control (31.1±4.5% CD31+, 20.5±2.5% CD144+) at the end of passage 5. Moreover, as iPSC-ECs overexpressing SIRT 1 continued to proliferate, the percentage of CD31+ cells increased from ˜60% at end of passage 5 to ˜90% at end of passage 7 to over 95% at end of passage 9.

### Functionality Assessment

Nitric oxide (NO) production was assessed via 4,5-diaminofluorescein.diacetate (DAF 2-DA) assay (Life Technologies). Cell mitogenic effect in response to VEGF (100 ng/ml) was assessed via MTT assay (Sigma-Aldrich) after treating cells in serum-free (starvation) medium, or starvation medium containing 100 ng/ml VEGF for 24 hours. Cell proliferation was determined by counting cell number every seven days after lentiviral transduction using a hemocytometer by excluding dead cells with Trypan blue. HDAC cell-based activity assay kit (Cayman Chemicals, Ann Arbor, Mich.) was used to assess iPSC-EC deacetylase activity due to SIRT1 by measuring the difference in HDAC activity between normal (0 μM Ex-527) and SIRT1 inhibited (10 μM Ex-527) culture conditions. All results were normalized to cell number via Alamar blue assay (Sigma-Aldrich).Cellular senescence assay kit (Cell Signaling Technology, Danvers, Mass.) was used to stain for senescence associated β-galactosidase (β-gal) in the presence or absence of Ex-527 (10 μM), a SIRT1 inhibitor, and the percentage of β-gal positive cells was quantified with ImageJ. SIRT1 overexpressing cells showed significantly higher NO production compared to control cells. VEGF stimulation led to increased mitogenicity in iPSC-ECs overexpressing SIRT1 but not control cells. The rate of cell proliferation was significantly increased at two passages following viral transduction with SIRT1 and these cells continued to proliferate throughout passage 10. In contrast, cells from the control group remained static after passage 5. Cell HDAC activity was significantly higher in cells with SIRT1 overexpression compared to the endogenous SIRT1 HDAC activity in cells with no viral control. Overexpression of SIRT1 led to a significant reduction of cells entering senescence when compared to control group (β-gal+ SIRT1 overexpressed: 3.4±2.7%, control: 38.6±3.3%, P<0.01)

### Flow Cytometry

EC markers CD31 (PECAM) and CD144 (VE-Cadherin) were stained with FITC-conjugated CD31 antibody (1:200 dilution, Sigma-Aldrich, St Lois, Mo.) and PE-conjugated CD144 antibody (1:200 dilution, Life Technologies, Carlsbad, Calif.). Flow cytometry was performed using BD LSR II flow cytometer (San Jose, Calif.) and the data analyzed with FlowJo analytical software (Ashland, Oreg.).

### Functional Analysis

Cellular senescence assay kit (Cell Signaling Technology, Danvers, Mass.) was used to stain for β-galactosidase (β-gal) in the presence or absence of Ex-527 (10 μM), a SIRT1 inhibitor (18), and the percentage of β-gal positive cells was quantified with ImageJ. HDAC cell-based activity assay kit (Cayman Chemicals, Ann Arbor, Mich.) was used to assess iPSC-EC deacetylase activity due to SIRT1 by measuring the difference in HDAC activity between normal (0 μM Ex-527) and SIRT1 inhibited (10 μM Ex-527) culture conditions. Cell proliferation was determined by counting cell number every seven days after lentiviral transduction using a hemocytometer by excluding dead cells with Trypan blue. Cell mitogenic effect in response to VEGF (100 ng/ml) was assessed via MTT assay (Sigma-Aldrich) after treating cells in serum-free (starvation) medium, starvation medium containing 100 ng/ml VEGF or regular growth medium for 24 hours. NO production was assessed via 4,5-diaminofluorescein.diacetate (DAF 2-DA) assay (Life Technologies). All results were normalized to cell number via Alamar blue assay (Sigma-Aldrich).

### EXAMPLE 2

### Results

### Influence of SIRT1 Overexpression on EC Phenotype

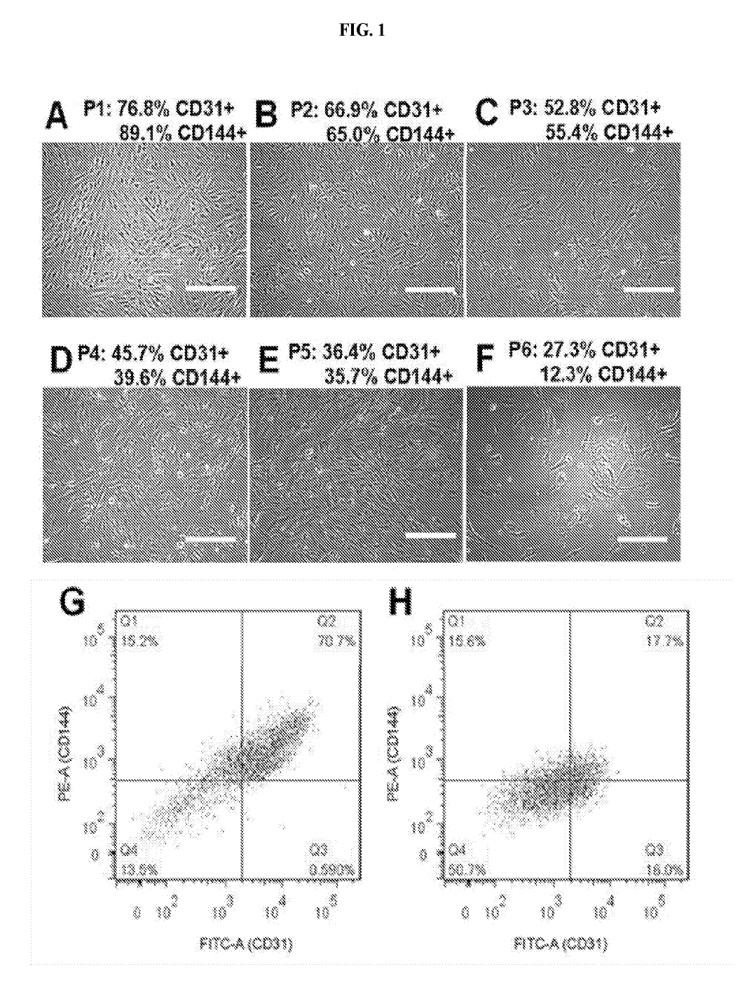

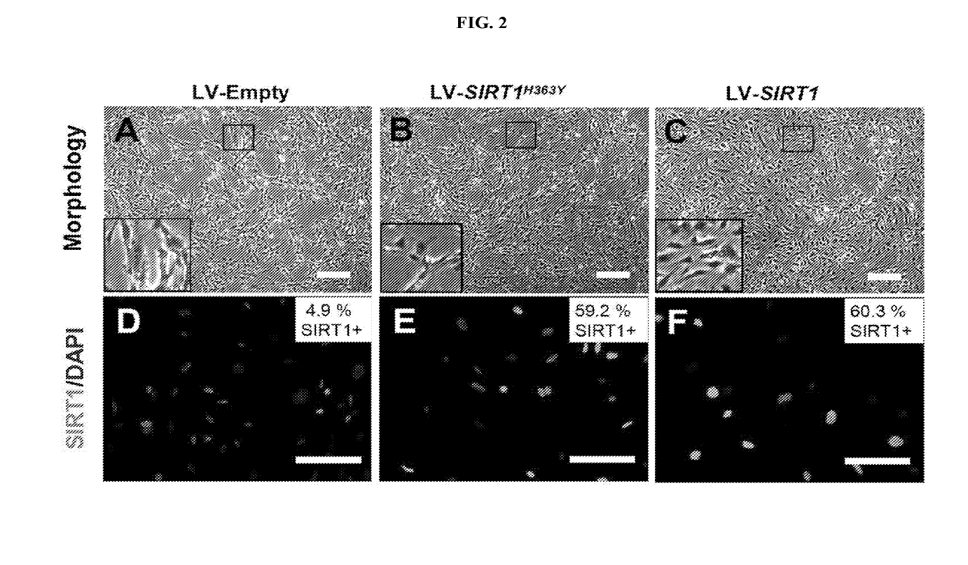

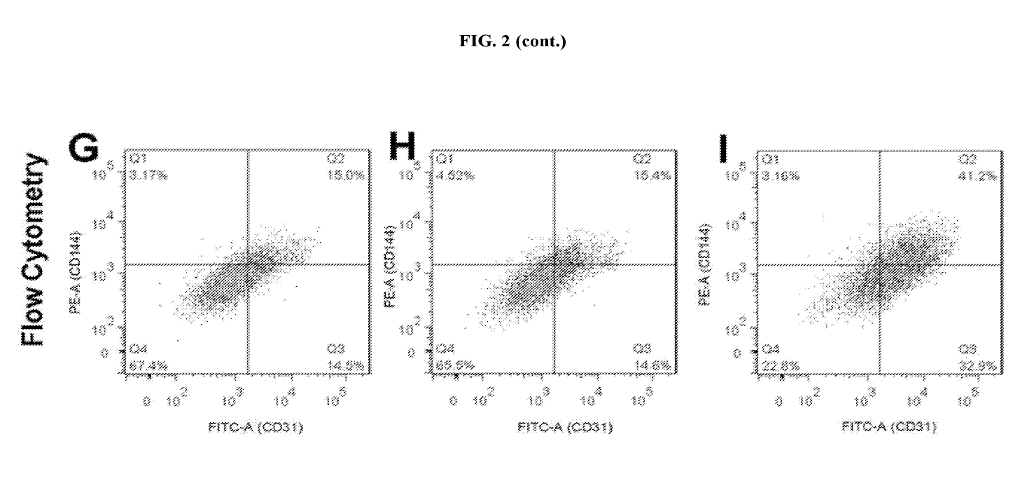

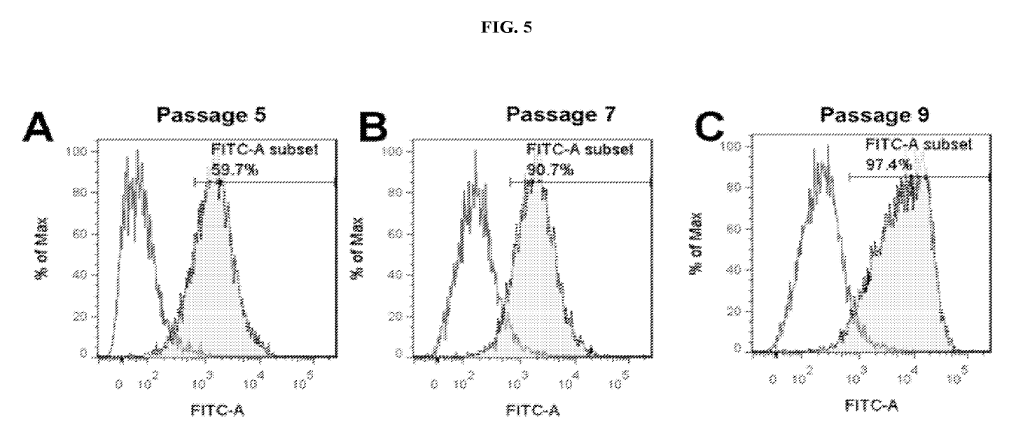

iPSC-ECs exhibited typical EC cobblestone-like morphology between passage 1 to 3, but gradually become fibroblast-like with decreased CD31 and CD144 expression over time (FIG. 1). A transduction efficiency of 60.3±7.3% was measured via immunohistomorphometry of SIRT1 positive cells. Cells overexpressing SIRT1 exhibit a higher degree of EC-like cobblestone morphology compared to the LV-empty and LV-SIRT1H363Y (FIG. 2). Expression of EC markers, such as CD31, were also significantly elevated in the LV-SIRT1group (61.8±3.6% CD31+) relative to controls (31.1±4.5% CD31+ for LV-empty and 39.8±15.4% CD31+ for LV-SIRT1H363Y) at the end of passage 5 (FIG. 2G-I). Moreover, as iPSC-ECs overexpressing SIRT1 continued to proliferate, the percentage of CD31+ cells increased from ˜60% at end of passage 5 to ˜90% at end of passage 7 to over 95% by end of passage 9 (FIG. 5).

### Effect of SIRT1 Overexpression on EC Function

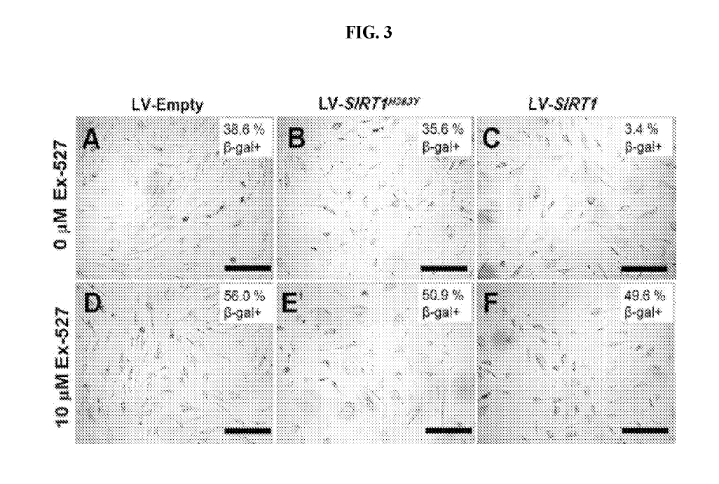

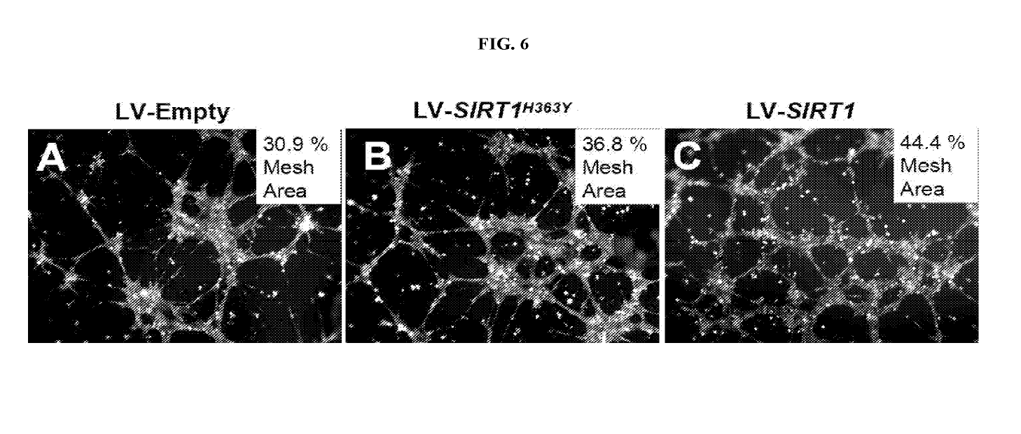

Overexpression of SIRT1 led to a significant reduction of cells entering senescence when compared to control groups (β-gal+ LV-SIRT1: 3.4±2.7%, LV-Empty: 38.6±3.3% and LV-SIRT1H363Y: 35.7±4.9%) (FIG. 3A-C). Blocking SIRT1 with the inhibitor Ex-527 led to a higher percentage of senescent cells in all groups, suggesting a contribution of endogenous SIRT1. (β-gal+ LV-SIRT1: 49.6±10.0%, LV-empty: 56.0±6.2%, and LV-SIRTH1H363Y: 50.9±6.8%) (FIG. 3D-F). Tube formation assay showed significantly denser and more organized vascular network formation for cells with SIRT1 overexpression (mesh area LV-SIRT1: 44.4±2.3%, LV-empty: 30.9±5.0%, and LV-SIRT1^{H363Y}: 36.8±3.5%) (FIG. 6), indicating improved angiogenesis potential.

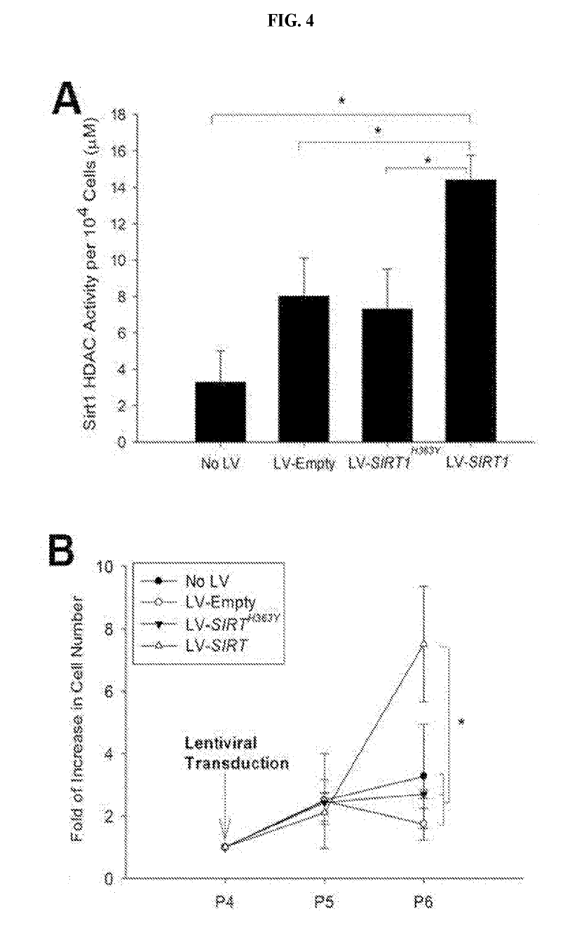

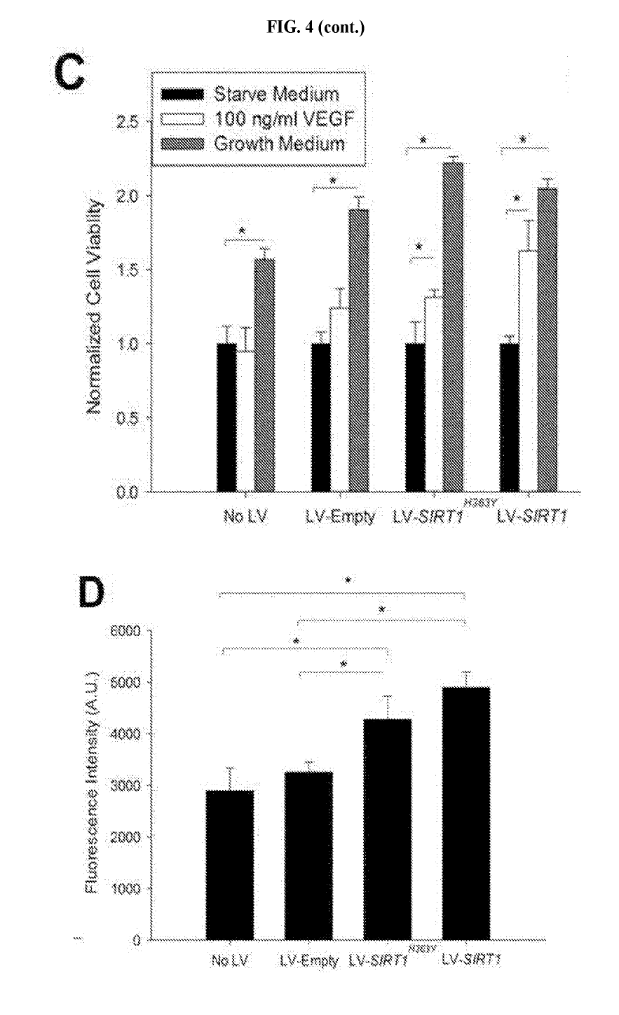

Cell HDAC activity was significantly higher in cells with LVSIRT1 compared to the endogenous SIRT1 HDAC activity in cells with no viral control, LV-Empty and LV-SIRT1H363Y (FIG. 4A). The rate of cell proliferation was significantly increased at two passages following viral transduction with LV-SIRT1 and these cells continued to proliferate throughout passage 10 (FIG. 4B). In contrast, cells from the control groups including LV-SIRT1H363Y remained static after passage 5. VEGF stimulation led to increased mitogenicity in iPSC-ECs overexpressing SIRT 1 (FIG. 4C) and these cells also showed significantly higher NO production compared to controls (FIG. 4D).

All publications and patents mentioned above and/or listed below are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

### REFERENCES

The following references, some of which are cited above by number, are herein incorporated by reference in their entireties.
1. Stepniewski J, Kachamakova-Trojanowska N, Ogrocki D et al. Induced pluripotent stem cells as a model for diabetes investigation. Sci Rep. 2015; 5.
2. Itzhaki I, Maizels L, Huber I et al. Modelling the long QT syndrome with induced pluripotent stem cells. Nature. 2011; 471:225-229.
3. Grskovic M, Javaherian A, Strulovici B et al. Induced pluripotent stem cells—opportunities for disease modelling and drug discovery. Nature Reviews Drug Discovery. 2011; 10:915-929.
4. Marchetto M C, Carromeu C, Acab A et al. A model for neural development and treatment of Rett syndrome using human induced pluripotent stem cells. Cell. 2010; 143:527-539.
5. Wu S M, Hochedlinger K. Harnessing the potential of induced pluripotent stem cells for regenerative medicine. Nature cell biology. 2011; 13:497-505.
6. Duan X, Tu Q, Zhang J et al. Application of induced pluripotent stem (iPS) cells in periodontal tissue regeneration. Journal of cellular physiology. 2011; 226:150-157.
7. Adams William J, Zhang Y, Cloutier J et al. Functional Vascular Endothelium Derived from Human Induced Pluripotent Stem Cells. Stem Cell Reports. 2013; 1:105-113.
8. Li Z, Hu S, Ghosh Z et al. Functional characterization and expression profiling of human induced pluripotent stem cell-and embryonic stem cell-derived endothelial cells. Stem cells and development 2011; 20:1701-1710.
9. Feng Q, Lu S-J, Klimanskaya I et al. Hemangioblastic Derivatives from Human Induced Pluripotent Stem Cells Exhibit Limited Expansion and Early Senescence. STEM CELLS. 2010; 28:704-712.
10. Cohen H Y, Miller C, Bitterman K J et al. Calorie restriction promotes mammalian cell survival by inducing the SIRT1 deacetylase. Science. 2004; 305:390-392.
11. Brunet A, Sweeney L B, Sturgill J F et al. Stress-dependent regulation of FOXO transcription factors by the SIRT1 deacetylase. Science. 2004; 303:2011-2015.
12. Potente M, Ghaeni L, Baldessari D et al. SIRT1 controls endothelial angiogenic functions during vascular growth. Genes & development 2007; 21:2644-2658.
13. Mattagajasingh I, Kim C-S, Naqvi A et al. SIRT1 promotes endothelium-dependent vascular relaxation by activating endothelial nitric oxide synthase. Proceedings of the National Academy of Sciences. 2007; 104:14855-14860.
14. Zu Y, Liu L, Lee M Y et al. SIRT1 promotes proliferation and prevents senescence through targeting LKB1 in primary porcine aortic endothelial cells. Circulation research. 2010; 106:1384-1393.
15. Zarzuelo M J, Lopez-Sepulveda R, Sanchez M et al. SIRT1 inhibits NADPH oxidase activation and protects endothelial function in the rat aorta: implications for vascular aging. Biochemical pharmacology. 2013; 85:1288-1296.
16. Potente M, Dimmeler S. Emerging roles of SIRT1 in vascular endothelial homeostasis. Cell Cycle. 2008; 7:2117-2122.
17. Jen M C, Baler K, Hood A R et al. Sustained, localized transgene expression mediated from lentivirus-loaded biodegradable polyester elastomers. Journal of Biomedical Materials Research Part A. 2013; 101A:1328-1335.
18. Solomon J M, Pasupuleti R, Xu L et al. Inhibition of SIRT1 catalytic activity increases p53 acetylation but does not alter cell survival following DNA damage. Molecular and cellular biology. 2006; 26:28-38.
19. Belair D G, Whisler J A, Valdez J et al. Human vascular tissue models formed from human induced pluripotent stem cell derived endothelial cells. Stem Cell Rev and Rep. 2014:1-15.
20. Moura R, Fadini G P, Tjwa M. Induced pluripotent stem (iPS) cells and endothelial cell generation: SIRT-ainly a good idea! Atherosclerosis. 2010; 212:36-39.
21. Ota H, Akishita M, Eto M et al. Sirt1 modulates premature senescence-like phenotype in human endothelial cells. Journal of molecular and cellular cardiology. 2007; 43:571-579.

### Sequences

SIRT1, Homo Sapiens
SEQ ID NO: 1
MIGTDPRTILKDLLPETIPPPELDDMTLWQIVINILSEPPKRKKRKDINT
IEDAVKLLQECKKIIVLTGAGVSVSCGIPDFRSRDGIYARLAVDFPDLPD
PQAMFDIEYFRKDPRPFFKFAKEIYPGQFQPSLCHKFIALSDKEGKLLRN
YTQNIDTLEQVAGIQRIIQCHGSFATASCLICKYKVDCEAVRGALFSQVV
PRCPRCPADEPLAIMKPEIVFFGENLPEQFHRAMKYDKDEVDLLIVIGSS
LKVRPVALIPSSIPHEVPQILINREPLPHLHFDVELLGDCDVIINELCHR
LGGEYAKLCCNPVKLSEITEKPPRTQKELAYLSELPPTPLHVSEDSSSPE
RTSPPDSSVIVTLLDQAAKSNDDLDVSESKGCMEEKPQEVQTSRNVESIA
EQMENPDLKNVGSSTGEKNERTSVAGTVRKCWPNRVAKEQISRRLDGNQY
LFLPPNRYIFHGAEVYSDSEDDVLSSSSCGSNSDSGTCQSPSLEEPMEDE
SEIEEFYNGLEDEPDVPERAGGAGFGTDGDDQEAINEAISVKQEVTDMNY
PSNKS
SIRT1, Mus musculus
SEQ ID NO: 2
MAAAAAAAAIGYRGPYTFVQQHLMIGTDPRTILKDLLPETIPPPELDDMT
LWQIVINILSEPPKRKKRKDINTIEDAVKLLQECKKIIVLTGAGVSVSCG
IPDFRSRDGIYARLAVDFPDLPDPQAMFDIEYFRKDPRPFFKFAKEIYPG
QFQPSLCHKFIALSDKEGKLLRNYTQNIDTLEQVAGIQRILQCHGSFATA
SCLICKYKVDCEAVRGDIFNQVVPRCPRCPADEPLAIMKPEIVFFGENLP
EQFHRAMKYDKDEVDLLIVIGSSLKVRPVALIPSSIPHEVPQILINREPL
PHLHFDVELLGDCDVIINELCHRLGGEYAKLCCNPVKLSEITEKPPRPQK
ELVHLSELPPTPLHISEDSSSPERTVPQDSSVIATLVDQATNNNVNDLEV
SESSCVEEKPQEVQTSRNVENINVENPDFKAVGSSTADKNERTSVAETVR
KCWPNRLAKEQISKRLEGNQYLFVPPNRYIFHGAEVYSDSEDDVLSSSSC
GSNSDSGTCQSPSLEEPLEDESEIEEFYNGLEDDTERPECAGGSGFGADG
GDQEVVNEAIATRQELTDVNYPSDKS
SIRT1 plasmid DNA (Addgene plasmid 1791)
SEQ ID NO: 3
AGCGCCTGATGCGGTATTTTCTCCTTACGCATCTGTGCGGTATTTCACAC
CGCATATCGACGCTCTCCCTTATGCGACTCCTGCATTAGGAAGCAGCCCA
GTAGTAGGTTGAGGCCGTTGAGCACCGCCGCCGCAAGGAATGGTGCATGC
AAGGAGATGGCGCCCAACAGTCCCCCGGCCACGGGGCCTGCCACCATACC
CACGCCGAAACAAGCGCTCATGAGCCCGAAGTGGCGAGCCCGATCTTCCC
CATCGGTGATGTCGGCGATATAGGCGCCAGCAACCGCACCTGTGGCGCCG
GTGATGCCGGCCACGATGCGTCCGGCGTAGAGGATCCAGACATGATAAGA
TACATTGATGAGTTTGGACAAACCACAACTAGAATGCAGTGAAAAAAATG
CTTTATTTGTGAAATTTGTGATGCTATTGCTTTATTTGTAACCATTATAA
GCTGCAATAAACAAGTTAACAACAACAATTGCATTCATTTTATGTTTCAG
GTTCAGGGGGAGGTGTGGGAGGTTTTTTAAAGCAAGTAAAACCTCTACAG
ATGTGATATGGCTGATTATGATCATTACTTATCTAGGTATAGGCTGCGCA
ACTGTTGGGAAGGGCGATCGGTGCGGGCCTCTTCGCTATTACGCCAGCTG
GCGAAAGGGGGATGTGCTGCAAGGCGATTAAGTTGGGTAACGCCAGGGTT
TTCCCAGTCACGACGTTGTAAAACGACGGCCAGTGAATTGTAATACGACT
CACTATAGGGCGAATTGAATTTAGCGGCCGCGAATTCGCCCTTGTCTAGA
GTGGAACAATTCCTGTACCTGCACAATTATTACACTATGATTTGTTTGAT
GGATAGTTCATGTCTGTTACTTCCTGTTTCACAGATATAGCTTCATTAAT
TGCCTCTTGATCATCTCCATCAGTCCCAAATCCAGCTCCTCCAGCTCTCT
CTGGAACATCAGGCTCATCTTCTAAGCCATTGTAGAATTCTTCAATTTCA
CTTTCATCCTCCATGGGTTCTTCTAAACTTGGACTCTGGCATGTCCCACT
ATCACTGTTACTGCCACAAGAACTAGAGGATAAGACGTCATCTTCAGAGT
CTGAATATACCTCAGCGCCATGGAAAATGTAACGATTTGGTGGCAAAAAC
AGATACTGATTACCATCAAGCCGCCTACTAATCTGCTCCTTTGCCACTCT
ATTAGGCCAGCATTTTCTCACTGTTCCAGCCACTGAAGTTCTTTCATTTT
TCTCCCCAGTACTAGAACCAACATTCTTCAAATCCGGATTTTCCATCTGT
TCAGCAATACTTTCAACATTCCTAGAAGTTTGTACTTCCTGTGGTTTTTC
TTCCATACAACCTTTTGATTCAGACACATCTAAATCATCATTACTCTTAG
CTGCTTGGTCTAAAAGTGTGACAATCACTGAAGAATCTGGTGGTGAAGTT
CTTTCTGGTGAACTTGAGTCTTCTGAAACATGAAGAGGTGTGGGTGGCAA
CTCTGACAAATAAGCCAATTCTTTTTGTGTTCGTGGAGGTTTTTCAGTAA
TTTCTGAAAGCTTTACAGGGTTACAGCAAAGTTTGGCATATTCACCACCT
AACCTATGACACAATTCATTAATTATGACATCACAGTCTCCAAGAAGCTC
TACATCAAAATGCAGATGAGGCAAAGGTTCTCTATTAATTAATATCTGAG
GCACTTCATGGGGTATGGAACTTGGAATTAGTGCTACTGGTCTTACTTTG
AGGGAAGACCCAATAACAATGAGGAGGTCAACTTCATCTTTGTCATACTT
CATGGCTCTATGAAACTGTTCTGGTAAATTTTCACCAAAAAACACAATCT
CTGGTTTCATGATAGCAAGCGGTTCATCAGCTGGGCACCTAGGACATCGA
GGAACTACCTGATTAAAAATATCTCCTCGTACAGCTTCACAGTCAACTTT
GTATTTACAAATCAGGCAAGATGCTGTTGCAAAGGAACCATGACACTGAA
TTATCCTTTGGATTCCCGCAACCTGTTCCAGCGTGTCTATGTTCTGGGTA
TAGTTGCGAAGTAGTTTTCCTTCCTTATCTGACAAGGCTATGAATTTGTG
ACAGAGAGATGGCTGGAATTGTCCAGGATATATTTCCTTTGCAAACTTGA
AGAATGGTCTTGGATCTTTTCTGAAATATTCAATATCAAACATCGCTTGA
GGATCTGGAAGATCTGGGAAGTCTACAGCAAGGCGAGCATAAATACCATC
CCTTGACCTGAAGTCAGGTATTCCACATGAAACAGACACCCCAGCTCCAG
TTAGAACTATAATTTTTTTGCACTCTTGCAGTAATTTCACAGCATCTTCA
ATTGTATTAATATCTTTTCTTTTTTTCCTTTTTGGTGGTTCTGAAAGGAT
ATTAATAACAATCTGCCACAGTGTCATATCATCCAACTCAGGTGGAGGTA
TTGTTTCCGGCAATAAATCTTTAAGAATTGTTCGAGGATCTGTGCCAATC
ATAAGATGTTGCTGAACAAAAGTATATGGACCTATCCGTGGCCTTGGAGT
CCAGTCACTAGAGCTTGCATGTGAGGCTCTATCCTCCTCATCACTTTCAC
AGGAATGAAAACCATTAGTGATAATTTCATCACCGAACAGAAGGTTATCT
CGGTACCCAATCGCCGCCGCCGCCGCCTCTTCCTCCTCCTCGCCCTCGTC
GTCGTCGTCTTCGTCGTACAAGTTGTCGGCCAGCGGTGGCTCCCGAGATG
GGCCCTGCAGGCCCGGCCCATTGTCTCCTTCCCCAGCCGCCGCAGTCGCC
TGGGCCTCTTGCTCCCCGCCTGCCGCCGCCGCCTCTGCCTCCGCCTCCCG
CCACAGCGCCGCCGCCGCCGCACCCGGGCAGCCCCTGGCCGCCGCCGGCA
CCTCACGCTCTGGGGCCGCCCCACCGGGCTCGCCCGGGCTCCGCTCGAGG
CCGGGACCATCTCTCCGCGGCCTCTTGCGGAGCGGCTCCCCGGCGGGGGA
CGACGCGGCCTCCCTGTCGGCCCCCGCCGCCGAGGGGGAGCCGCCGGGCT
GAAGGGCGAGGGCCGCCTCGTCCGCCGAATTCCCGGGTTTATCGTCATCG
TCCTTGTAGTCCATCCTGAGGATCCGAGCTCGGTACCAAGCTTAGATCTC
CTCCAAAAAAGCCTCTTCACTACTTCTGGAATAGCTCAGAGGCCGAGGCG
GCCTCGGCCTCTGCATAAATAAAAAAAATTAGTCAGCCATGGGGCGGAGA
ATGGGCGGAACTGGGCGGAGTTAGGGGCGGGATGGGCGGAGTTAGGGGCG
GGACTATGGTTGCTGACTAATTGAGATGCATGCTTTGCATACTTCTGCCT
GCTGGGGAGCCTGGGGACTTTCCTTGCTGACTAATTGAGATGCATGCTTT
GCATACTTCTGCCTGCTGGGGAGCCTGGGGACTTTCCACACCCTAACTGA
CACACATTCCACAGCTGGTTCTTTCCGCCTCAGAAGGTCAGGTGGCACTT
TTCGGGGAAATGTGCGCGGAACCCCTATTTGTTTATTTTTCTAAATACAT
TCAAATATGTATCCGCTCATGAGACAATAACCCTGATAAATGCTTCAATA
ATATTGAAAAAGGAAGAGTATGAGTATTCAACATTTCCGTGTCGCCCTTA
TTCCCTTTTTTGCGGCATTTTGCCTTCCTGTTTTTGCTCACCCAGAAACG
CTGGTGAAAGTAAAAGATGCTGAAGATCAGTTGGGTGCACGAGTGGGTTA
CATCGAACTGGATCTCAACAGCGGTAAGATCCTTGAGAGTTTTCGCCCCG
AAGAACGTTTTCCAATGATGAGCACTTTTAAAGTTCTGCTATGTGGCGCG
GTATTATCCCGTGTTGACGCCGGGCAAGAGCAACTCGGTCGCCGCATACA
CTATTCTCAGAATGACTTGGTTGAGTACTCACCAGTCACAGAAAAGCATC
TTACGGATGGCATGACAGTAAGAGAATTATGCAGTGCTGCCATAACCATG
AGTGATAACACTGCGGCCAACTTACTTCTGACAACGATCGGAGGACCGAA
GGAGCTAACCGCTTTTTTGCACAACATGGGGGATCATGTAACTCGCCTTG
ATCGTTGGGAACCGGAGCTGAATGAAGCCATACCAAACGACGAGCGTGAC
ACCACGATGCCTGCAGCAATGGCAACAACGTTGCGCAAACTATTAACTGG
CGAACTACTTACTCTAGCTCCCGGCAACAATTAATAGACTGGATGGAGGC
GGATAAAGTTGCAGGACCACTTCTGCGCTCGGCCCTTCCGGCTGGCTGGT
TTATTGCTGATAAATCTGGAGCCGGTGAGCGTGGGTCTCGCGGTATCATT
GCAGCACTGGGGCCAGATGGTAAGCCCTCCCGTATCGTAGTTATCTACAC
GACGGGGAGTCAGGCAACTATGGATGAACGAAATAGACAGATCGCTGAGA
TAGGTGCCTCACTGATTAAGCATTGGTAACTGTCAGACCAAGTTTACTCA
TATATACTTTAGATTGATTTAAAACTTCATTTTTAATTTAAAAGGATCTA
GGTGAAGATCCTTTTTGATAATCTCATGACCAAAATCCCTTAACGTGAGT
TTTCGTTCCACTGAGCGTCAGACCCCGTAGAAAAGATCAAAGGATCTTCT
TGAGATCCTTTTTTTCTGCGCGTATCTGCTGCTTGCAAACAAAAAAACCA
CCGCTACCAGCGGTGGTTTGTTTGCCGGATCAAGAGCTACCAACTCTTTT
TCCGAAGGTAACTGGCTTCAGCAGAGCGCAGATACCAAATACTGTCCTTC
TAGTGTAGCCGTAGTTAGGCCACCACTTCAAGAACTCTGTAGCACCGCCT
ACATACCTCGCTCTGCTAATCCTGTTACCAGTGGCTGCTGCCAGTGGCGA
TAAGTCGTGTCTTACCGGGTTGGACTCAAGACGATAGTTACCGGATAAGG
CGCAGCGGTCGGGCTGAACGGGGGGTTCGTGCACACAGCCCAGCTTGGAG
CGAACGACCTACACCGAACTGAGATACCTACAGCGTGAGCTATGAGAAAG
CGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGG
TATCTTTATAGTCCTGTCGGGTTTCGCCACCTCTGACTTGAGCGTCGATT
TTTGTGATGCTCGTCAGGGGGGCGGAGCCTATGGAAAAACGCCAGCAACG
CGGCCTTTTTACGGTTCCTGGCCTTTTGCTGGCCTTTTGCTCACATGTTC
TTTCCTGCGTTATCCCCTGATTCTGTGGATAACCGTATTACCGCCTTTGA
GTGAGCTGATACCGCTCGCCGCAGCCGAACGACCGAGCGCAGCGAGTCAG
TGAGCGAGGAAGCGGAAG

1. A composition comprising induced pluripotent stem cell (iPSC)-derived endothelial cells (ECs) that overexpress Sirtuin1 (SIRT1).

2. The composition of claim 1, wherein the iPSC-derived ECs comprise exogenous nucleic acid encoding SIRT1.

3. The composition of claim 2, wherein the exogenous nucleic acid encodes a polypeptide having at least 70% sequence identity with all or a portion of wild-type human SIRT1 (SEQ ID NO: 1).

4. The composition of claim 2, wherein the exogenous nucleic acid encoding SIRT1 is within an expression vector.

5. The composition of claim 4, wherein the expression vector is a lentiviral vector.

6. A method of maintaining endothelial cell (EC) phenotype, improving EC function, enhancing proliferative capacity, and/or overcoming early senescence in induced pluripotent stem cell (iPSC)-derived ECs, comprising overexpressing Sirtuin1 (SIRT1) in said iPSC-derived ECs.

7. The method of claim 6, wherein overexpressing SIRT1 in said iPSC-derived ECs comprises tranducing, transfecting, or transforming a SIRT1-encoding vector into the iPSC-derived ECs.

8. The method of claim 7, wherein the SIRT1-encoding vector is tranduced, transfected, or transformed into the iPSC-derived ECs after passage 4.

9. A composition comprising transplantable cells, tissue, or organ comprising the iPSC-derived ECs of claim 1.

10. A method of treating a subject comprising administering the composition of claim 9.

11. A method of testing an agent comprising administering the agent to a composition of claim 1.

12. The method of claim 11, wherein the agent is testing for therapeutic efficacy.

13. The method of claim 11, wherein the agent is testing for toxicity.

14. A method comprising:
(a) inducing the formation of pluripotent stem cells (iPSCs) from non-pluripotent somatic cells;
(b) differentiating the iPSCs into iPSC-derived endothelial cells (ECs); and
(c) overexpressing Sirtuin1 (SIRT1) in said iPSC-derived ECs.
